(19) United States
(12) Patent Application Publication
(10) Pub. No.: US 2024/0001980 A1
(43) Pub. Date: Jan. 4, 2024

(54) Multidirectional Tilting Mechanism and a Method of Operating the Same

(71) Applicant: Sartorius Stedim North America Inc., Bohemia, NY (US)

(72) Inventors: Peter Smit, Mount Sinai, NY (US); Joseph Deacutis, Coram, NY (US); Alexander Halt, Wehretal (DE)

(73) Assignee: Sartorius Stedim North America Inc., Bohemia, NY (US)

(21) Appl. No.: 17/855,920

(22) Filed: Jul. 1, 2022

(51) Int. Cl. B62B 3/08; B62B 3/02; B67D 7/84; F16C 11/04

(52) U.S. Cl. CPC B62B 3/08; B62B 3/02; B67D 7/84; F16C 11/04; B62B 2206/003

(57) ABSTRACT

A tilting mechanism including a base, a tilting deck, four hinges, and a lifting member. Each hinge is positioned adjacent a corner of the base and the tilting deck. Each hinge includes a globe and a receiver with the globe or the receiver secured to the base frame or the deck frame and the other secured to the other of the base or the tilting deck. Each hinge includes a locking member that selectively secures a globe within a respective receiver. The lifting member is secured at a center of the base frame and a center of the deck frame. The lifting member is configured to urge the tilting deck away from the base such that selective engagement allows for tilting of the tilting deck relative to the base in at least four directions.

### BACKGROUND

### 1. Technical Field

The present disclosure relates to fluid handling and, more specifically, to a multidirectional tilting mechanism for filling and draining containers.

### 2. Discussion of Related Art

During manufacture, storage, and distribution of biopharmaceutical compositions, containers are filled and drained of biopharmaceutical compositions in a liquid state. For example, a biopharmaceutical composition is pumped from a production vessel into one or more storage containers. Once filled, the storage containers may be frozen, stored, and/or shipped to another facility. Before use, the biopharmaceutical composition within the storage containers is thawed and/or drained from the storage containers.

In some applications, containers, e.g., Celsius® FFT containers from Sartorius AG, are loaded into a Fill and Drain Station (FDS) that is used to fill and drain the containers. In the FDS the containers are held in a horizontal orientation. In the horizontal orientation, not all of a biopharmaceutical composition can be drained from the containers. In addition, once the container is drained, the tubing from the container to the fill and drain station may include additional biopharmaceutical composition. In addition, in the horizontal orientation, it may be difficult to purge air from the tubing leading to the container and/or the container itself prior to filling of the container.

It has been found that tilting the containers during filling and/or draining may allow for a more complete fill or drain of a biopharmaceutical composition to and from the container. A currently available tilting mechanism 910 maybe capable of tilting in two-directions, e.g., leftward and rightward, is shown in FIG. 1. The tilting mechanism 910 includes a base 915 that supports a tilting platform 920 and a linear actuator 930. The linear actuator 930 is operably coupled to the tilting platform 920 by a linkage 940 to actuate the tilting platform 920 to the right or the left. During testing, it has been found that the tilting mechanism 910 is complex and prone to premature failure. In addition, the tilting mechanism 910 is limited to tilting the platform 920 to the right or the left. Further, the components of the tilting mechanism 910 and in particular, the linear actuator 930, may be cost prohibitive. In some applications, the tilting mechanism 910 with a FDS secured thereto may have a height which prevents the tilting mechanism 910 and the FDS from passing through a traditional doorway.

### SUMMARY

This disclosure relates generally to a tilting mechanism that is capable of tilting a weighing station/platform of an FDS in multiple directions. The disclosed tilting mechanism may have a simplified construction to previous solutions, e.g., tilting mechanism 910. In addition, the disclosed tilting mechanism may have a reduced cost relative to the tilting mechanism 910. In some embodiments, the tilting mechanism may allow for tilting of the FDS in four directions, e.g., forward, backward, a first or rightward side direction, and a second or leftward side direction. In certain embodiments, the tilting mechanism may allow for tilting in eight directions including the directions between each of the forward, backward, the first side direction, and the second side direction. The disclosed tilting mechanism may have a reduced height compared to prior art tilting mechanisms, e.g., the tilting mechanism 910, such that the disclosed tilting mechanism may fit through a standard doorway with an FDS supported on the disclosed tilting mechanism.

In use, the disclosed tilting mechanism may be used to prime fill lines with biopharmaceutical composition prior to filling the containers or may be used to drain containers filled with biopharmaceutical composition. Priming air from fill lines may prevent air from entering the containers such the that entire container is filled with a biopharmaceutical composition. In some embodiments, it may be beneficial to tilt containers at an angle in a range of 5 degrees to 45 degrees. The disclosed tilting mechanism may allow for tilting in a plurality of directions during filling and draining by operating a single lifting element to urge a tilting deck away from a fixed base based on a state of a plurality of hinges selectively securing the tilting deck to the fixed base.

In an embodiment of the present disclosure, a tilting mechanism includes a base, a tilting deck, four multi-dimensional hinges, and a lifting member. The base includes a rectangular base frame and the tilting deck includes a rectangular deck frame. The hinges are positioned with one hinge adjacent each corner of the base frame and the deck frame. Each frame includes a globe and a receiver with the globe or the receiver secured to the base frame or the deck frame and the other of the globe or the receiver secured to the other of the base frame or the deck frame. Each hinge includes a locking member such that the hinge has a catch state in which the locking member secures a globe within a respective receiver and a release state in which the locking member allows a globe to enter and exit the respective receiver. The lifting member is secured at a center of the base frame and a center of the deck frame. The lifting member is configured to urge the deck frame away from the base frame such that selective engagement of the hinges allows for tilting of the deck frame relative to the base frame in at least four directions.

In embodiments, the tilting deck may be configured to support a weighing platform that is configured to determine an amount of fluid disposed within one or more containers disposed on the weighing platform. The lifting member may be a lifting bag that expands in response to receiving fluid therein. The lifting bag may be an airbag that expands in response to receiving gas therein. The lifting bag may be in fluid communication with a pump. The pump may provide fluid to expand the bag.

In some embodiments, the tilting deck may have a neutral point when the lifting member is in a non-extended positon in which each globe is disposed in a respective one of the receivers.

In certain embodiments, the tilting deck may have a first tilted position in which a first hinge and a fourth hinge of the four hinges are in the catch state and a second hinge and a third hinge of the four hinges are in the release state such that the tilting deck is tilted about a first tilting axis defined through the first hinge and the fourth hinge in response to extension of the lifting member. In the first tilted position, the tilting deck may be rotated about the first tilting axis at an angle in a range of 5 degrees to 45 degrees. The tilting deck may have a second tilted position in which the first hinge and the fourth hinge are in the release state and the second hinge and the third hinge are in the catch state such that the tilting deck is tilted about a second tilting axis defined through the second hinge and the third hinge in response to extension of the lifting member. The tilting deck may have a third tilted position in which the first hinge and the second hinge are in the catch state and the third hinge and the fourth hinge are in the release state such that the tiling deck is tilted about a third tilting axis defined through the first hinge and the second hinge in response to extension of the lifting member.

In particular embodiments, the locking member may be a fork that is linearly slideable into and out of a receiver to transition the hinge between the catch state and the release state. The fork may be linearly actuated by a linear actuator, a solenoid, or a pneumatic actuator. The hinge may include a biasing member that urges the fork into or out of the receiver.

In another embodiment of the present disclosure, a fill and draining system includes a platform, a valve tower, and a tilting mechanism. The platform configured to support at least one container to be filled or drained. The valve tower is configured to distribute fluid to each container of the at least one container. The tilting mechanism may be any of the tilting mechanisms detailed herein. The tilting mechanism supports the platform on a tilting deck thereof.

In another embodiment of the present disclosure, a multi-dimensional hinge includes a globe, a receiver, and a locking member. The receiver is configured to selective receive the globe. The locking member is linearly translatable between a tach position in which the globe is secured within the receiver and a release position in which the globe is allowed to enter and exit the receiver. The receiver is movable in at least two degrees of freedom relative to the globe when the globe is secured within the receiver. The locking member may be a fork with two tongs that are linearly translatable into and out of the respective receiver to secure the globe within the respective receiver. The hinge may include a biasing member that is configured to urge the locking member towards the catch position or the release position.

In another embodiment of the present disclosure, a tilting mechanism includes a base, a tilting deck, and a lifting member. The base has a base frame and the tilting deck has a deck frame. The lifting member is secured at a center of the base frame and a center of the deck frame. The lifting member is configured to urge the tilting deck away from the base deck such that the tilting deck is tillable relative to the base in at least four directions.

In embodiments, the lifting member is an extendable bag disposed between the base frame and the deck frame. The tilting mechanism may include a multi-dimensional hinge disposed at each corner of the base frame and the deck frame. Each multi-dimensional hinge having a globe secured to one of the base frame or the deck frame and receiver secured to the other of the base frame and the deck frame. The receiver may be configured to selective secure a respective globe therewithin.

In another embodiment of the present disclosure, a method of operating a tilting mechanism for filling or draining a fluid container supported on the tilting mechanism includes tilting the deck frame supporting a container in a first direction by placing a first hinge and a second hinge in a catch state and a third hinge a fourth hinge in a release state and actuating a lifting mechanism to urge the deck frame away from a base frame. The method also includes tilting the deck frame in a second direction, opposite the first direction, by placing the first hinge and the second hinge in a release state and the third hinge the fourth hinge in a catch state and actuating the lifting mechanism to urge the deck frame away from the base frame.

In embodiments, the method may include tiling the deck frame in a third direction different from the first direction and the second direction by placing the first hinge and the third hinge in a catch state and the second hinge and the fourth hinge in a release state and actuating the lifting mechanism to urge the deck frame away from the base frame.

In some embodiments, actuating the lifting mechanism includes providing a fluid into a lifting bag such that the lifting bag expands to urge the deck frame away from the base frame. The tilting deck frame in the first direction may include the deck frame rotating about a first axis that is defined through the first hinge and the second hinge. Tilting the deck frame in the second direction may include the deck frame rotating about a second axis that is defined through the third hinge and the fourth hinge.

In certain embodiments, placing one of the first, second, third, or fourth hinge in the catch state comprises actuating a locking mechanism to secure a globe within a receiver such that the hinge is rotatable in two-degrees of freedom about the globe. Actuating the locking mechanism may include translating a fork into the receiver such that two tongs of the fork are disposed about a neck of the globe to prevent the globe from withdrawing from within the receiver.

In particular embodiments, placing one of the first, second, third, or fourth hinge in the release state includes actuating a locking mechanism to allow a globe to exit a receiver. Actuating the locking mechanism to allow the globe to exit a receiver may include translating a fork such that tongs of the fork are disposed substantially outside of the receiver such that the globe is capable of withdrawing from the receiver.

Further, to the extent consistent, any of the embodiments or aspects described herein may be used in conjunction with any or all of the other embodiments or aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a perspective view of a prior art tilting mechanism;

FIG. 2 is a schematic view of a filling system provided in accordance with an embodiment of the present disclosure;

FIG. 3A is a schematic view of a draining system provided in accordance with an embodiment of the present disclosure in a neutral position;

FIG. 3B is a schematic view of the draining system of FIG. 3A in a tilted position;

FIG. 4 is a top, perspective view of a tilting mechanism provided in accordance with an embodiment of the present disclosure including a fixed base and a tilting deck in a neutral position;

FIG. 5 is a bottom, perspective view of the tilting deck of FIG. 4;

FIG. 6 is a top, perspective view of the fixed base of FIG. 4;

FIG. 7 is a perspective view of a multi-dimensional hinge of the tilting mechanism of FIG. 4 in a release state;

FIG. 8 is a perspective view of the multi-dimensional hinge of the tilting mechanism of FIG. 4 in a catch state;

FIG. 9 is a top, perspective view of the tilting mechanism of FIG. 4 with the tilting deck tilted in a first direction;

FIG. 10 is a top, perspective view of the tilting mechanism of FIG. 4 with the tilting deck tilted in a second direction;

FIG. 11 is a top, perspective view of the tilting mechanism of FIG. 4 with the tilting deck tilted in a third direction;

FIG. 12 is a top, perspective view of the tilting mechanism of FIG. 4 with the tilting deck tilted in a fourth direction;

FIG. 13 is a flow chart of a method of filling a container in accordance with an embodiment of the present disclosure; and

FIG. 14 is flow chart of a method of draining a container in accordance with an embodiment of the present disclosure.

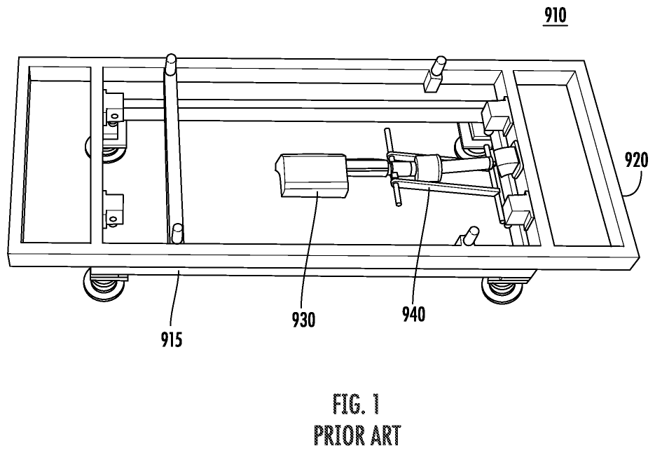

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like. Further, as used herein the term “biopharmaceutical compositions” refers to a product coming from biotechnology, culture environments, cell cultures, buffer solutions, artificial nutrition liquids, blood products and derivatives of blood products, a pharmaceutical product, or more generally a product intended to be used in the medical field including, without any limitation, monoclonal antibodies (mAbs), therapeutic proteins, viruses, lipid nanoparticles, vaccines, virus banks, exosomes, cell banks, and cell therapy products.

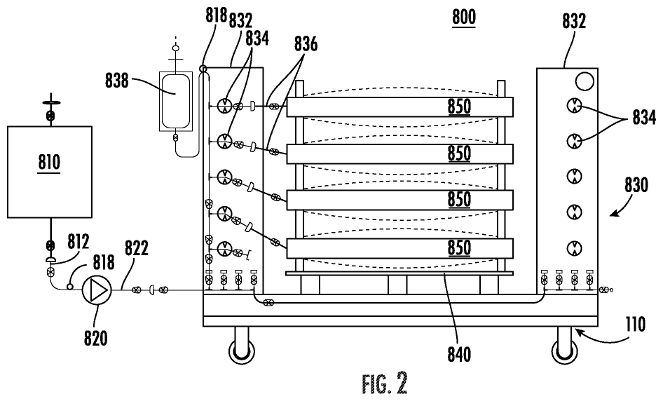

Referring now to FIG. 2, a schematic of a filling system 800 is provided in accordance with an embodiment of the present disclosure. The filling system 800 includes a supply vessel 810, a supply pump 820, and a fill and drain station (FDS) 830. The supply vessel 810 includes a biopharmaceutical composition to be distributed to one or more containers secured in the FDS 830. The supply vessel 810 is in fluid communication with the FDS 830 via tubing 812, 822 that passes through the supply pump 820. The filling system 800 may include sterilizing filters, buffer supply, and buffer waste.

The FDS 830 includes a platform 840 that receives a pallet or structure including one or more containers 850 to be filled or drained. The platform 840 may be a weighing platform that measures a weight of a biopharmaceutical composition within the containers 850. The platform 840 may be a pallet with multiple containers 850 supported above a base. As shown, the pallet is a euro-sized pallet that supports four 75 liter containers 850 for a total of 300 liters of biopharmaceutical compositions to be filled and/or drained into or from the containers 850. In use, the pallet may be loaded and/or unloaded onto the platform with machinery such as a forklift. In some embodiments, the pallet may have a capacity to support ten containers 850 having a volume of 12, 6, 4, or 2 liters each. The pallet may have a capacity to support between one and ten containers having a volume from 2 L to 75 L each. In some embodiments, the pallet may have a capacity greater than 300 L and/or to support more than ten containers.

The FDS 830 includes at least one distribution column or valve tower 832 on a side of the FDS 830 that receives a biopharmaceutical composition from the pump 820. In certain embodiments, a single valve tower 832 is used of the FDS 830, e.g., when large containers 850 are used. In some embodiments, the FDS 830 may include a valve tower 832 on each side of the FDS 830. Each valve tower 832 may include a valve 834 for each container 850 on the platform 840 to place the container 850 in fluid communication with the pump 820 via tubing 836. When each valve tower 832 is used, e.g., when small containers 850 are supported on the platform 840, a valve 834 of a first valve tower 832 may place a first container 850 in fluid communication with the pump 820 and another valve 834 of a second valve tower 832 may place a second container 850 in fluid communication with the pump 820. As shown, the valves 834 for a respective container 850 may be positioned slightly above the respective container 850 such that fluid flow into the container 850 may be aided by gravity. In some embodiments, the platform 840 may be tilted to position the respective valve 834 above the respective container 850 to assist in priming the filling tubes 836, e.g., assist gasses in flowing out of the tubes towards a bubble trap or air purge bag 838. In embodiments with a valve tower 832 on each side of the FDS 830, the platform 840 may be tilted in a first direction to prime filling tubes 836 by placing valves 834 of a first valve tower 832 above respective containers 850 and tilted in a second direction to prime filling tubes 836 by placing valves 834 of a second valve tower 832 above respective containers 850.

The valve towers 832 may include the air purge bag 838 that is configured to receive gases from the FDS 830 and/or the filling tubes 836. The gases may flow from the tubing 812, 822 connecting the supply vessel 810 with the FDS 830 and/or may flow from the filling tubes 836. In some embodiments, the filling tubes 836 are connected at the valves 834 with a tilted T-connection such that the filling tube 836 has an angle of less than 90 degrees on a lower side of the connection and an angle of greater than 90 degrees on an upper side of the connection. In some embodiments, the tilted T-connection is tilted in a range of 10 degrees to 45 degrees, e.g., 15 degrees. The tilted T-connection may assist in allowing gases to flow towards the air purge bag 838.

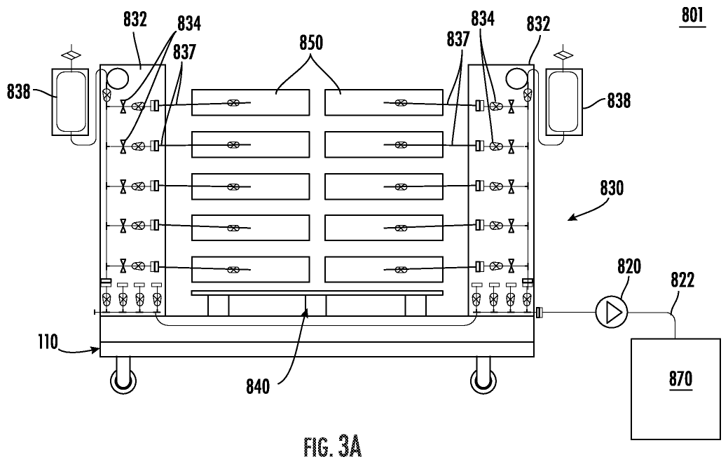

With reference to FIGS. 3A and 3B, a schematic of a draining system 801 is provided in accordance with an embodiment of the present disclosure. Elements of the draining system 801 may be similar to the filling system 800 and represented with identical labels with only the differences detailed herein for brevity. The draining system 801 includes an FDS 830, containers 850, a pump 820, and a product vessel 870.

In a draining configuration, the FDS 830 includes the valve towers 832 with the valves 834. The valve 834 is placed in fluid communication with a respective container 850 via tubing 836. The valve 834 for a respective container 850 may be position slightly below the respective container 850 such that fluid flow from the container 850 may be aided by gravity. The valve tower 832 may include a purge bag 838 to capture gases from the valve tower 832, the tubing 837, and/or the containers 850. Similar to the filling process detailed above, the platform 840 may be tilted in a first direction to position valves 834 of a first valve tower 832 below respective containers 850 and in a second direction to position valves 834 of a second valve tower 832 below respective containers 850 as shown in FIG. 3B.

The pump 820 is in fluid communication with the FDS 830 and the product vessel 870 by tubing 822. The tubing 822 may include sample containers that allow for sampling of a biopharmaceutical composition being distributed periodically during the draining operation. The pump 820 may pump the biopharmaceutical composition from the containers 850 to the product vessel 870.

In some embodiments, the product vessel 870 may be replaced with a distribution system including a plurality of product vessels or containers connected to one or more distribution hubs. Non-limiting examples of distribution systems are described in U.S. Pat. No. 11,319,201 and U.S. Patent Publication Nos. 2021/0024338, 2021/0188615, and 2021/0395071, the entire contents of each of these patents and publications are hereby incorporated by reference.

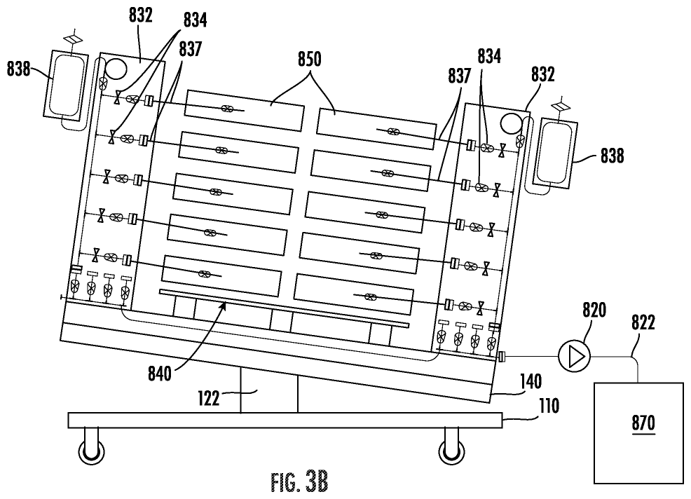

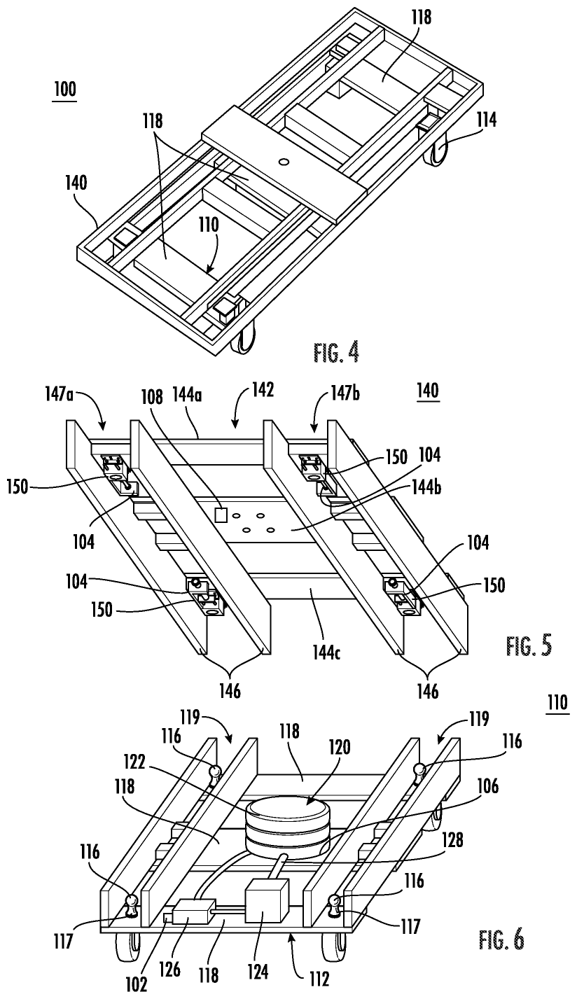

Referring to FIGS. 4-6, a tilting mechanism 100 is provided in accordance with the present disclosure. The tilting mechanism 100 may support the entire FDS 830 or the platform 840 of the FDS 830. The tilting mechanism 100 includes a fixed base 110 and a tilting deck 140. In some embodiments, the fixed base 110 may support the valve towers 832 of the FDS 830 and the tilting deck 140 may support the platform 840 of the FDS 830. In some embodiments, the fixed base 110 is supported by one or more legs that include sensors to determine a weight supported by the leg such that the fixed base 110 is a weighing platform. For example, the fixed base 110 may

The fixed base 110 includes a base frame 112 that may be fixed in position or may include wheels or rollers 114 that allow the base frame 112 to be moved. One or more of the wheels or rollers 114 may include a locking mechanism that allow the base frame 112 to be fixed in position during a filling or draining operation. The fixed base 110 includes a globe 116 at each corner of the fixed base 110 and extending upwards towards the tilting deck 140. The fixed base 110 may include one or more cross members 118 that extend between the sides of the fixed base 110. One of the cross members 118 may be disposed substantially at the center of the fixed base 110. The fixed base 110 may include channels 119 that are formed of vertical sidewalls that run perpendicular to the cross members 118 with the globes 116 disposed within the channels 119.

The fixed base 110 also includes a lifting system 120 having a lifting element 122 disposed at or near a center of the fixed base 110. The lifting element 122 is configured to provide a lifting force in the middle of the tilting deck 140. The lifting element 122 is secured to the fixed base 110 and the tilting deck 140 in a manner that the lifting element 122 may expand in a generally vertical direction regardless of the orientation of the tilting deck 140 relative to the fixed base 110. The lifting element 122 and/or the attachment points of the lifting element 122 are configured to articulate at different angles such that as the lifting element 122 extends to urge the tilting deck 140 away from the fixed base 110 the tilting deck 140 tilting in a variety of directions based on the state of multi-dimensional hinges 170 of the tilting mechanism 100.

As shown, the lifting element 122 is a lifting bag. The lifting element 122 may be an air bag or hydraulic bag that expands in response to an increased pressure of fluid within the lifting element 122. The lifting element 122 may be connected to a supply of compressed air. For example, the lifting element 122 may be connected to a facility compressed air line to receive compressed air to inflate the lifting element 122 such that the lifting element 122 expands to urge the center of the tilting deck 140 away from the fixed base 110. The lifting element 122 may include a release valve 128 for releasing air from within the lifting element 122 into the environment. As the air provided to the lifting element 122 is facility compressed air, releasing air from within the lifting element 122 may not compromise a clean room environment.

In certain embodiments, the fixed base 110 includes a pump 126 to provide fluid to the lifting element 122 such that the lifting element 122 expands to urge the center of the tilting deck 140 away from the fixed base 110. In some embodiments, the pump 126 may draw air from the environment and the lifting element 122 may include a release valve 128 for releasing air into the environment. In certain embodiments, the lifting system 120 is a closed system including the lifting element 122, a reservoir or expansion tank 124, and the pump 126. In such embodiments, the pump 126 may draw fluid from the expansion tank 124 to pressurize the lifting element 122 and the release valve 128 releases fluid back to the expansion tank 124. A closed system may allow for the lifting system 120 to be operated in a clean room environment without compromising the environment from the release of fluid from the lifting element 122.

With particular reference to FIG. 5, the tilting deck 140 includes a deck frame 142 that includes cross supports 144 and channel members 146 that run between and perpendicular to the cross supports 144 such that the deck frame 142 is substantially rectangular in shape. The tilting deck 140 may be slightly larger than the fixed base 110 such that the tilting deck 140 fits over the fixed base 110. In some embodiments, the tilting deck 140 may be smaller than the fixed base 110 such that the tilting deck 140 is disposed within the fixed base 110. In embodiments where the tilting deck 140 is smaller than the fixed base 110, the fixed base 110 may support stationary portions of a FDS, e.g., valve towers, and the tilting deck 140 may support a tilting platform 840 of the FDS. Two of the cross supports 144a, c may be positioned adjacent the ends of the deck frame 142 with each cross support 144 overlying two of the globes 116 of the fixed base 110. A central cross support 144b of the cross supports 144 may be positioned at a central position of the deck frame 142 to cover a center point of the deck frame 142. The central cross support 144b may be secured to a top end of the lifting element 122. The channel members 146 may extend vertically from the cross supports 144 to define a first channel 147a and a second channel 147b adjacent the sides of the deck frame 142. The channel members 146 may act as stops to limit the downward movement of the tilting deck 140 relative to the fixed base 110.

The tilting deck 140 includes a receiver 150 at each corner of the deck frame 142. Each receiver 150 is positioned to receive a respective one of the globes 116. In some embodiments, the receivers 150 are positioned within the first channel 147a or the second channel 147b of the deck frame 142. Each receiver 150 is sized to receive one of the globes 116 therein and to selectively catch and release a respective one of the globes 116.

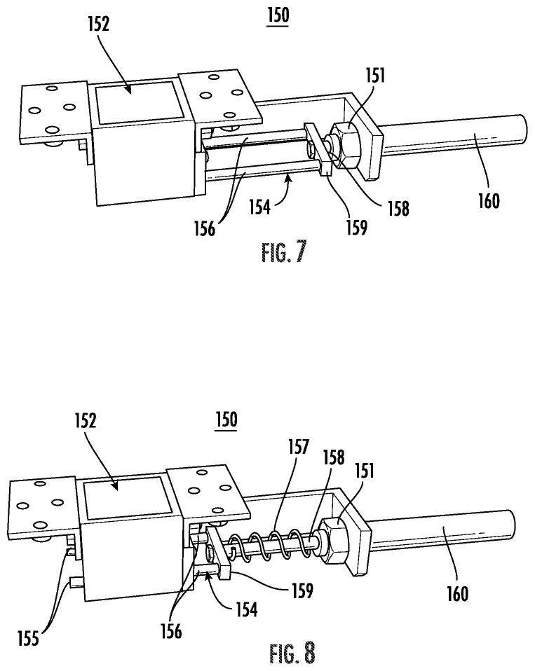

Continuing to refer to FIGS. 7 and 8, the receivers 150 define a chamber 152 and a locking member or fork 154. The chamber 152 is sized and dimensioned to receive a respective one of the globes 116 therein. The locking fork 154 has a first or released state (FIG. 7) and a second or catch state (FIG. 8). In the released state, the fork 154 is disposed substantially outside of the chamber 152 such that the globe 116 is free to enter and exit the chamber 152. In the released position, tips 155 of the fork 154 may be partially disposed in the chamber 152 but in a position that does not interfere with movement of the globe 116 into or out of the chamber 152. In the catch state, the fork 154 is inserted into the chamber 152 such that tongs 156 of the fork 154 are disposed around a neck 117 of a globe 116 disposed within the chamber 152 such that the globe 116 is secured within the chamber 152. The chamber 152 may include a lining or bearing surface that is shaped to complement a shape of the globe 116 such that the chamber 152 can pivot about the globe 116 to form a multi-dimensional hinge 170 to allow selective movement of the tilting deck 140 relative to the fixed base 110 as detailed below. When the globe 116 is secured within the chamber 152, the receiver 150 is moveable relative to the globe in at least two-degrees of freedom.

The fork 154 may include a shank 158 that extends from the bridge 159 that interconnects the tongs 156. The receiver 150 may include a stop 151 that is abutted by the bridge 159 in the released position to prevent additional retraction of the fork 154 beyond the released state. The shank 158 may be actuated by an actuator 160. The actuator 160 may be actuated by a variety of actuators including, but not limited to, a linear actuator, a solenoid, a hydraulic actuator, an electromagnetic actuator, and a pneumatic actuator. In some embodiments, the actuator 160 operates in a similar manner or by a similar medium to the lifting member or element 122 such that a medium is shared between the lifting element 122 and the actuators 160. For example, the lifting element 122 may be an air bag and the actuator 160 may receive air from the same pump or facility air supply to move the fork 154 between the catch state and the release state. In some embodiments, the actuator may be a dual acting pneumatic cylinder such that the fork 154 is moved towards the catch state in response to air entering through a first valve and the fork 154 is moved towards the release state in response to air entering through a second valve. A dual acting pneumatic cylinder may allow the fork 154 to stay in a previous state until an input is provided. In certain embodiments, the fork 154 includes a biasing member 157 about the shank 158 that biases the fork 154 towards the catch state or the release state. For example, the biasing member 157 may be a spring that biases the fork 154 towards the catch state or that biases the fork towards the release state. In such embodiments, the actuator 160 moves the fork 154 against the bias of the biasing member 157 to move the fork 154 towards the catch state or the release state.

As shown herein, each multi-dimensional hinge 170 is formed with a single receiver and a single globe. In some embodiments, each multi-dimensional hinge 170 is a hinge assembly including one or more single dimensional hinges. For example, a single multi-dimensional hinge may be formed of two single degree of freedom hinges with each single degree of freedom hinge having a catch state and a release state.

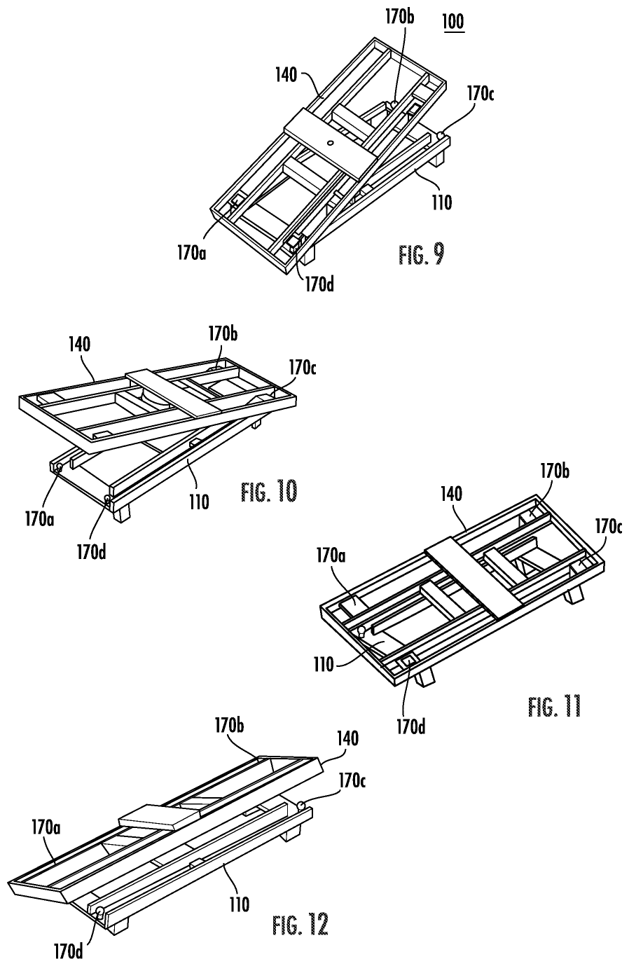

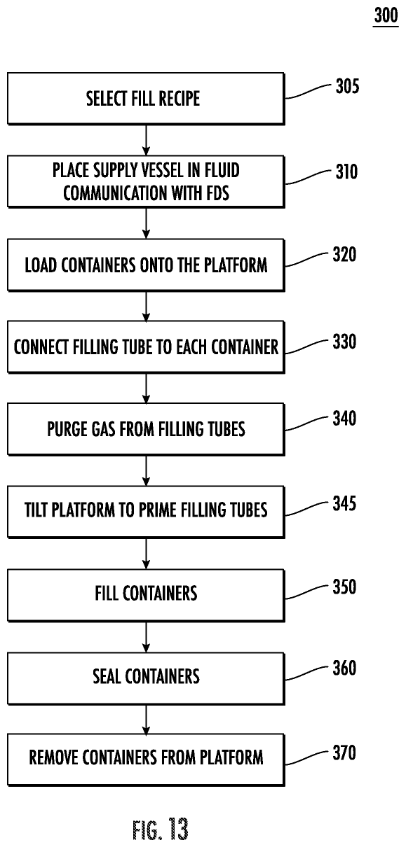

Referring now to FIGS. 9-12, the lifting element 122 and the hinges 170 are operated in a manner according to embodiments of the present disclosure to actuate the tilting deck 140 between a natural or flat position of FIG. 5 to a leftward tilt position (FIG. 9), a rightward tilt position (FIG. 10), a forward tilt position (FIG. 11), and a backward tilt position (FIG. 12). For example, as shown in FIG. 9, the hinges 170a and 170d are in the catch state and the hinges 170b and 170c are in the release state such that when the lifting element 122 is actuated towards an extended position, the tilting deck 140 pivots about a first tilting axis that passes through the hinges 170a and 170d to move towards a leftward tilt position. As shown in FIG. 10, the hinges 170b and 170c are in the catch state and the hinges 170a and 170d are in the release state such that when the lifting element 122 is actuated towards an extended position, the tilting deck 140 pivots about a second tilting axis that passes through the hinges 170b and 170c to move towards the rightward tilt position. As shown in FIG. 11, the hinges 170c and 170d are in the catch state and the hinges 170b and 170c are in the release state such that when the lifting element 122 is actuated towards an extended position, the tilting deck 140 pivots about a fourth tilting axis that passes through the hinges 170c and 170d to move towards a backward tilt position. As shown in FIG. 12, the hinges 170a and 170b are in the catch state and the hinges 170c and 170d are in the release state such that when the lifting element 122 is actuated towards an extended position, the tilting deck 140 pivots about a third tilting axis that passes through the hinges 170a and 170b to move towards a forward tilt position.

While not explicitly shown, the lifting element 122 and the hinges 170 may be operated to tilt the tilting deck 140 to positions between the leftward tilt position, the rightward tilt position, the forward tilt position, and the backward tilt position. For example, the hinge 170a may be in the catch state with the hinges 170b, c, d in the release state such that when the lifting element 122 is extended the tilting deck 140 is tilted to a left, forward tilt position.

As described above, the globes 116 are secured to the fixed base 110 and the receivers 150 are secured to the tilting deck 140; however, in some embodiments, one or more of the globes 116 may be secured to the tilting deck 140 and one or more of the receivers 150 may be secured to the fixed base 110.

The angle of the tilt of the tilting deck 140 to the left, right, forward, and backward tilt positions is controlled by the distance that the lifting element 122 extends towards the extended position. The amount or degree of tilt to a respective direction may be selected from a number of preset degrees, e.g., 5, 10, 15, 20, 25, 30, 35, 40, and 45 degrees, or may be variable. In some embodiments, the tilting mechanism 100 includes a controller 102 (FIG. 6) and one or more sensors 104, 106, 108 to determine a state of the hinges 170, the lifting element 122, and the angle of the tilting deck 140. The controller 102 may move the tilting deck 140 in response to user input or may move the tilting deck 140 based on a preprogrammed operation, e.g., prime and fill, prime and drain. For example, sensors 104 (FIG. 5) may be configured to determine a state of each of the hinges 170, the sensor 106 (FIG. 6) may determine an amount of extension or lift of the lifting element 122, and the sensor 108 (FIG. 5) may determine a direction and/or tilt angle of the tilting deck 140.

In some embodiments, the tilting mechanism 100 may include one or more safety sensors to prevent over tilting and/or tipping of the weighing station. The safety sensors may prevent people from pinch and/or crush hazards from tilting of the tilting deck 140. The safety sensors may detect people entering a hazard area around the weighing station before or during tilting. When one of the safety sensors is activated, tilting may be prevented from starting or may be stopped. The safety sensors may include one or more laser safety scanners that are configured to detect a person entering a tilting or hazard zone before or during tilting of the tilting deck 140. In some embodiments, the safety sensors may include, but not be limited to, light curtains, pressure sensitive mats, Lidar, or ultrasonic sensors. In certain embodiments, the tilting mechanism 100 may include a fence system around the tilting mechanism 100 to prevent persons or objects from entering the hazard or tilting zone during tilting. The fence system may include an interlocked gate that when opened, prevents or stops tilting of the tilting deck 140.

In some embodiments, the controller 102 may detect a state of each hinge 170 before tilting to prevent extension of the lifting element 122 if one or more of the hinges 170 are out of position. The controller 102 may monitor an angle of tilt of the tilting deck 140 and prevent the tilting deck 140 from tilting beyond a preset maximum tilt angle, e.g., 15, 20, 25, 30, 45, 40, or 45 degrees. The tilting mechanism 100 may include mechanical stops to limit tilting to prevent the tilting deck 140 lifting beyond a mechanical limit.

Referring now to FIG. 13, a method of filling a container is described in accordance with an embodiment of the present disclosure with reference to the filling system 800 of FIG. 2 and the tilting mechanism of FIGS. 4-12 and is referred to generally as method 300. To begin a filling operation, a fill recipe is chosen (Step 305). The fill recipe may be selected by interfacing with the controller 102. With the fill recipe chosen, a supply vessel 810 including a biopharmaceutical composition is placed in fluid communication with a FDS 830 with tubing 812, 822 (Step 310). The tubing 812, 822 may include one or more filters that are primed once the supply vessel 810 is placed in fluid communication with the FDS 830.

The containers 850 are loaded onto the platform 840 of the FDS 830 (Step 320). The containers 850 may be loaded onto the platform 840 as part of a bulk shipper. The bulk shipper may be a euro sized pallet that includes between one and ten containers 850. With the containers 850 loaded onto the platform, a filling tube 836 is connected between a respective valve 834 and a respective container 850 (Step 330).

When all the containers 850 are connected to a respective valve 834 by a respective filing tube 836, the filling tubes 836 are primed to purge air from the filling tubes 836 (Step 340). The filling tubes 836 are primed by tilting the platform 840 such that the valves 834 are positioned above the respective container 850 and the valves 834 are opened such that gasses within the filling tubes 836 are replaced with biopharmaceutical composition (Step 345). The containers 850 may include a closure, e.g., a clamp or a pinch valve, to close the containers 850 until the respective filling tube 836 is primed. Priming the containers may take between thirty seconds and five minutes, e.g., one minute and forty-five seconds. In some embodiments, the filling tubes 836 may be primed simultaneously or may be primed sequentially.

In embodiments, the platform 840 may need be tilted in a first direction to prime filling tubes 836 of a first valve tower 832 and tilted a second direction to prime filling tubes 836 of a second valve tower 832. In certain embodiments, the platform 840 may need to be tilted in more than two directions to prime all the filling tubes 836. For example, 75 L Celsius® FFT containers have a filling and draining port disposed on the left side of the container such that the tilting deck 140 is tilted rightward or clockwise to prime the container and the filling tubes 836. In contrast, 12 L, 6 L, 4 L, and 2 L Celsius® FFT containers have a filling and draining port on the front of the container such that the tilting deck 140 is tilted backward to prime the containers and the filling tubes 836. In embodiments, where both valve towers 832 are used with filling and draining ports of the containers on each side of the platform 840, the tilting deck 140 may be tilted rightward to prime the containers and filling tubes 836 on the left valve tower 832 and then the tilting deck 140 may be tilted leftward to prime the containers and the filing tubes 836 on the right valve tower 832.

Once the filling tubes 836 are primed, the containers 850 are placed in fluid communication with the valves 834 such that the containers 850 are filled (Step 350). The containers 850 may be filled simultaneously or sequentially. The amount of the biopharmaceutical composition may be measured by a weight of the platform 840. As noted above, the platform 840 may be a weighing platform such that the amount of a biopharmaceutical composition can be determined by the weight of the platform 840. The tilting deck 140 may be returned to a neutral position after priming and during filling. During priming and/or filling, the pump 820 may be activated to flow a biopharmaceutical composition from the supply vessel 810 to the containers 850.

The containers 850 may be filled in a tilted or a neutral position. When the containers 850 are filled in a neutral position, the containers 850 may be tilted at the end of a filling process to purge a biopharmaceutical composition from the filling tubes 836 and into the containers 850.

With the containers 850 filled and the filling tubes 836 purged, the containers 850 are sealed (Step 360) and the bulk shipper, including the containers 850, is removed from the platform 840 (Step 370).

It has been found that priming the containers and filling tubes 836 prior to filling, gas within the containers 850 can be reduced or eliminated. For example, in some experiments, the amount of air within each container can be reduced by 100 mL. Reducing air in the containers may increase a life of biopharmaceutical compositions stored within the containers. The increase of life of the biopharmaceutical composition may be a result of reduced interactions between the biopharmaceutical compositions and remaining air within the container.

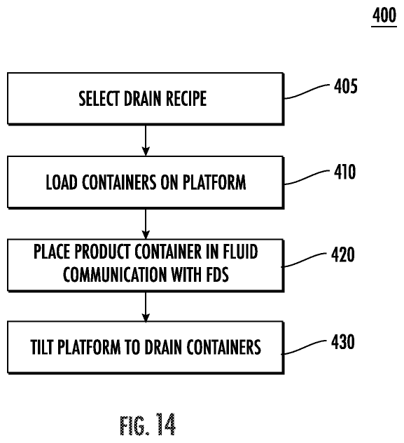

Referring now to FIG. 14, a method of draining a container is described in accordance with an embodiment of the present disclosure with reference to the draining system 801 of FIG. 3 and the tilting mechanism of FIGS. 4-12 and is referred to generally as method 400. To begin a draining operation, a drain recipe is selected (Step 405). The containers 850 including a biopharmaceutical composition to be drained are loaded onto the platform 840 (Step 410). The containers 850 may be loaded onto the platform as part of bulk shipper. A product vessel 870 for receiving a biopharmaceutical composition is placed in fluid communication with a FDS 830 with tubing 822 (Step 420). With the product vessel 870 placed in fluid communication with the FDS 830, one or more of the container valves 834 are opened to allow a biopharmaceutical composition to flow from the containers 850 to the product vessel 870 (Step 420). The pump 820 may be activated to flow the biopharmaceutical composition from the containers 850 into the product vessel 870. During draining, the tilting mechanism 100 may be in a neutral position or may be tilted as detailed below. When one or more of the containers 850 are drained or substantially drained the container 850 is tilted to purge the container 850 and the draining tubes 837 connected to the container 850 (Step 430). Tilting the container 850 and the draining tube 837 allows for a substantially complete draining of the container 850 and the draining tube 837.

During draining it may be necessary to tilt the platform 840 in more than one direction to purge the containers 850 and the draining tube 837. For example, 75 L Celsius® FFT containers have a filling and draining port disposed on the left side of the container such that the tilting deck 140 is tilted to the leftward or counter-clockwise to drain the containers 850 and the draining tube 837. In contrast, 12 L, 6 L, 4 L, and 2 L Celsius® FFT containers have a filling and draining port on the front of the container such that the tilting deck 140 is tilted forward to drain the containers and the draining tubes 837. In embodiments, where both valve towers 832 are used with filling and draining ports of the containers on each side of the platform 840, the tilting deck 140 may be tilted leftward to purge the containers 850 and the draining tubes 837 on the left valve tower 832 and then the tilting deck 140 may be tilted rightward to purge the containers and the draining tubes 837 on the right valve tower 832. In some configurations, without tilting during a draining process, a limited amount of a biopharmaceutical composition can be drained from a container 850. As such, tilting the containers 850 may allow for a substantially complete draining of a container 850. Tilting the containers 850 and the draining tubes 837 may allow for additional biopharmaceutical composition to be extracted from the containers 850.

In some embodiments, steps of the filling method 300 and/or the draining method 400 may be automated such that the controller 102 controls the pump 820, the valves 834, and/or the tilting mechanism 100. In addition, the controller 102 may monitor an amount of a biopharmaceutical composition that is filled or drained from the containers 850 and switch between containers 850 during a filling or draining operation. In some embodiments, the controller 102 may operate valves or clamps of the containers 850 during the filling and/or draining operation. In certain embodiments, the controller 102 may operate valves of sampling tubes during a filling and/or draining operation to sample a biopharmaceutical composition at different times during the respective operation.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A tilting mechanism comprising:
a base having a rectangular base frame;
a tilting deck having a rectangular deck frame;
four multi-dimensional hinges with one hinge positioned adjacent each corner of the base frame and the deck frame, each hinge comprising a globe and a receiver with the globe or the receiver secured to the base frame or the deck frame and the other of the globe or the receiver secured to the other of the base frame or the deck frame, each hinge further comprising a locking member such that each hinge has a catch state in which the locking member secures a globe within a respective receiver and a release state in which the locking member allows a globe to enter and exit the respective receiver; and
a lifting member secured at a center of the base frame and a center of the deck frame, the lifting member configured to urge the deck frame away from the base frame such that selective engagement allows for tilting of the deck frame relative to the base frame in at least four directions.

2. The tilting mechanism according to claim 1, wherein the tilting deck is configured to support a weighing platform, the weighing platform configured to determine an amount of fluid disposed within one or more containers disposed on the weighing platform.

3. The tilting mechanism according to claim 1, wherein the lifting member is a lifting bag that expands in response to receiving fluid therein.

4. The tilting mechanism according to claim 3, wherein the lifting bag is an airbag that expands in response to receiving gas therein.

5. The tilting mechanism according to claim 3, wherein the lifting bag is in fluid communication with a pump, the pump providing fluid to expand the lifting bag.

6. The tilting mechanism according to claim 1, wherein the tilting deck has a neutral position when the lifting member is in a non-extended position in which each globe is disposed in a respective one of the receivers.

7. The tilting mechanism according to claim 1, wherein the tilting deck has a first tilted position in which a first hinge and a fourth hinge of the four hinges are in the catch state and a second hinge and a third hinge of the four hinges are in the release state such that the tilting deck is tilted about a first tilting axis defined through the first hinge and the fourth hinge in response to extension of the lifting member.

8. The tilting mechanism according to claim 7, wherein in the first tilted position, the tilting deck is rotated about the first tilting axis at an angle in a range of 5 degrees to 45 degrees.

9. The tilting mechanism according to claim 7, wherein the tilting deck has a second tilted position in which the first hinge and the fourth hinge are in the release state and the second hinge and the third hinge are in the catch state such that the tilting deck is tilted about a second tilting axis defined through the second hinge and the third hinge in response to extension of the lifting member.

10. The tilting mechanism according to claim 9, wherein the tilting deck has a third tilted position in which the first hinge and the second hinge are in the catch state and the third hinge and the fourth hinge are in the release state such that the tilting deck is tilted about a third tilting axis defined through the first hinge and the second hinge in response to extension of the lifting member.

11. The tilting mechanism according to claim 1, wherein the locking member is a fork that is linearly slideable into and out of a respective receiver to transition the hinge between the catch state and the release state.

12. The tilting mechanism according to claim 11, wherein the fork is linearly actuated by a linear actuator, a solenoid, or a pneumatic actuator.

13. The tilting mechanism according to claim 11, wherein the hinge includes a biasing member that urges the fork into or out of the receiver.

14. A fill and draining system comprising:
a platform configured to support at least one container to be filled or drained;
a valve tower configured to distribute fluid to each container of the at least one container; and
a tilting mechanism according to claim 1, the tilting mechanism supporting the platform on a tilting deck thereof.

15. A multi-dimensional hinge comprising:
a globe;
a receiver configured to selectively receive the globe; and
a locking member linearly translatable between a catch position in which the globe is secured within the receiver and a release position in which the globe is allowed to enter and exit the receiver, the receiver movable in at least two degrees of freedom relative to the globe when the globe is secured within the receiver.

16. The hinge according to claim 15, wherein the locking member is a fork with two tongs that are linearly translatable into and out of the receiver to secure the globe within the receiver.

17. The hinge according to claim 15, further comprising a biasing member configured to urge the locking member towards the catch position or the release position.

18. A tilting mechanism comprising:
a base having a base frame;
a tilting deck having a deck frame; and
a lifting member secured at a center of the base frame and a center of the deck frame, the lifting member configured to urge the tilting deck away from the base frame such that the tilting deck is tillable relative to the base in at least four directions.

19. The tilting mechanism according to claim 18, wherein the lifting member is an extendable bag disposed between the base frame and the deck frame.

20. The tilting mechanism according to claim 18, further comprising multi-dimensional hinge disposed at each corner of the base frame and the deck frame, each multi-dimensional hinge selectively engageable to allow the deck frame to tilt relative to the base frame in at least two-degrees of freedom.

21. The tilting mechanism according to claim 21, wherein the multi-dimensional hinge includes a globe secured to one of the base frame or the deck frame and a receiver secured to the other of the base frame and the deck frame, the receiver configured to selectively secure a respective globe therewithin.

22. A method of operating a tilting mechanism for filling or draining a fluid container supported on the tilting mechanism, the method comprising:
tilting a deck frame supporting a container in a first direction by placing a first hinge and a second hinge in a catch state and a third hinge and a fourth hinge in a release state and actuating a lifting mechanism to urge the deck frame away from a base frame; and
tilting the deck frame in a second direction, opposite the first direction, by placing the first hinge and the second hinge in a release state and the third hinge and the fourth hinge in a catch state and actuating the lifting mechanism to urge the deck frame away from the base frame.

23. The method according to claim 22, further comprising tilting the deck frame in a third direction different from the first direction and the second direction by placing the first hinge and the third hinge in a catch state and the second hinge and the fourth hinge in a release state and actuating the lifting mechanism to urge the deck frame away from the base frame.

24. The method according to claim 22, wherein actuating the lifting mechanism includes providing a fluid into a lifting bag such that the lifting bag expands to urge the deck frame away from the base frame.

25. The method according to claim 22, wherein tilting the deck frame in the first direction includes the deck frame rotating about a first axis defined through the first hinge and the second hinge, wherein tilting the deck frame in the second direction includes the deck frame rotating about a second axis defined through the third hinge and the fourth hinge.

26. The method according to claim 22, wherein placing one of the first, second, third, or fourth hinge in the catch state comprises actuating a locking mechanism to secure a globe within a receiver such that the hinge is rotatable in two-degrees of freedom about the globe.

27. The method according to claim 26, wherein actuating the locking mechanism to secure the globe includes translating a fork into the receiver such that two tongs of the fork are disposed about a neck of the globe to prevent the globe from withdrawing from within the receiver.

28. The method according to claim 22, wherein placing one of the first, second, third, or fourth hinge in the release state comprises actuating a locking mechanism to allow a globe to exit a receiver.

29. The method according to claim 28, wherein actuating the locking mechanism to allow the globe to exit a receiver includes translating a fork such that tongs of the fork are disposed substantially outside of the receiver such that the globe is capable of withdrawing from the receiver.
